(19) United States
(12) Patent Application Publication
(10) Pub. No.: US 2023/0062798 A1
(43) Pub. Date: Mar. 2, 2023

(54) MIMICKING PHYSIOLOGIC CONDITIONS DURING ORGAN OR TISSUE PERFUSION

(71) Applicant: Medtronic Ireland Manufacturing Unlimited Company, Dublin (IE)

(72) Inventors: Thomas Fernando Valenzuela, Cloverdale, CA (US); Emma A. Schinstock, St. Paul, MN (US); Paul Anthony Iaizzo, White Bear Lake, MN (US); Stefan S. Tunev, Santa Rosa, CA (US)

(21) Appl. No.: 17/820,728

(22) Filed: Aug. 18, 2022

Related U.S. Application Data
(60) Provisional application No. 63/237,968, filed on Aug. 27, 2021.

(51) Int. Cl. A01N 1/02; C12M 1/00

(52) U.S. Cl. CPC A01N 1/0247; C12M 29/10; A01N 1/0284; A01N 1/0268; A01N 1/0252

(57) ABSTRACT

A system is configured to perfuse one or more organs and/or associated tissues while monitoring the physiological function, testing medical devices or therapies, or both. The system includes a pump, a container, a fluid circuit, a platform, and at least one actuator. The pump is configured to generate a flow of a fluid. The container defines an interior cavity and is configured to receive at least one organ or tissue and maintain at least one environmental condition associated with the at least one organ or tissue within the interior cavity. The fluid circuit is configured to fluidically couple the pump to the at least one organ or tissue. The platform is operably coupled to the container. The at least one actuator is configured to move the platform to mimic at least one biological movement associated with the at least one organ or tissue.

This application claims the benefit of U.S. Provisional Application No. 63/237,968, filed Aug. 27, 2021, the entire content of which is incorporated herein by reference.

### TECHNICAL FIELD

This disclosure generally relates to systems for perfusing one or more organs and/or associated tissues while monitoring physiological function, testing medical devices or therapies, or both.

### BACKGROUND

Perfusion is the passage of fluid through the circulatory system or lymphatic system to an organ or a tissue. For example, perfusion may indicate a flow of blood that transports oxygen or a metabolic substance to an organ through a capillary network. In general, tissues require an adequate blood supply for health and life, and poor perfusion (e.g., malperfusion) may cause health problems, such as cardiovascular disease, including coronary artery disease, cerebrovascular disease, peripheral artery disease, etc.

A system may be configured to permit investigations of the physiological conditions of a completely isolated organ, such as a mammalian heart. Such a system may include forcing a perfusate (i.e., oxygen carrying fluid), such as blood, through a coronary vasculature of the organ (e.g., by means of a catheter), which in turn may provoke one or more physiological reactions that reflect the function of the organ.

### SUMMARY

In some examples, a system includes a pump configured to generate a flow of a fluid; a container defining an interior cavity configured to receive at least one organ or tissue and maintain at least one environmental condition associated with the at least one organ or tissue within the interior cavity; a fluid circuit configured to fluidically couple the pump to the at least one organ or tissue; a platform operably coupled to the container; and at least one actuator configured to move the platform to mimic at least one biological movement associated with the at least one organ or tissue.

In some examples, a method of operating a perfusion system includes generating, by a pump, a flow of a fluid through a fluid circuit, wherein the pump comprises a pump outlet, and wherein the fluid circuit is configured to fluidically couple the pump to at least one organ or tissue such that the fluid flows through the at least one organ or tissue in response to the pump generating the flow of the fluid, wherein the perfusion system comprises a container defining an interior cavity configured to receive the at least one organ or tissue, and wherein the container is configured to maintain at least one environmental condition associated with the at least one organ or tissue within the interior cavity; and moving, by an actuator operably coupled to a platform operably coupled to the container, the platform to mimic at least one biological movement associated with the at least one organ or tissue.

Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating an example system configured to induce one or more isolated organs to mimic at least one physiologic condition, in accordance with examples of this disclosure.

FIG. 2 is a conceptual diagram illustrating an example container, in accordance with one or more examples of this disclosure.

FIG. 3 is a conceptual diagram illustrating an organ positioned within an interior cavity of an example container, in accordance with examples of this disclosure.

FIG. 4 is a conceptual diagram of control circuitry for controlling components of an example system, in accordance with examples of this disclosure.

FIG. 5 is a conceptual diagram of a system configured to enable visualization of organ, in accordance with examples of this disclosure.

FIG. 6 is flow diagram of an example technique for operating a system, in accordance with examples of this disclosure.

### DETAILED DESCRIPTION

Isolated organs (e.g., isolated mammalian organs), such as kidneys, livers, spleens, hearts, lungs, skeletal muscles, eyes, gallbladders, or the like, and/or associated tissues may be used to determine the efficacy of novel or developing medical devices and therapies, train clinicians for using medical devices or therapies, or the like. Such testing and training can be costly, thereby limiting the number of new devices and therapies that can be evaluated, particularly beyond initial development phases, and the number of clinicians who can be trained in using new medical devices or techniques. Further, the value of the data gathered from the testing and the knowledge gained through the training may be limited based on how accurately the isolated organs mimic physiology of intact organs. Accordingly, this disclosure is directed to systems configured to induce one or more isolated organs and/or associated tissues to mimic physiologic conditions, in turn allowing for device and/or therapy investigations and refinements in a timely and cost-effective manner with greater fidelity to patient physiology and responses. Further, such systems may promote hands-on clinician training with functioning organs and mimicked physiological conditions, in this way offering more realistic and effective training.

The systems described herein are configured to reanimate, within a container (e.g., a micro-environment), one or more organs. The system may be configured to deliver a perfusate (e.g., blood or a blood substitute, such as a Krebs-Henseleit buffer) to the one or more organs and/or associated tissues via a pump, which the system may control to mimic (e.g., replicate, reproduce, imitate, simulate, or the like) a variety of conditions, such as pulse rates, stroke volumes, blood pressures, or the like. The perfusate may maintain the viability of organs, associated tissues, and other structures. The system also includes one or more actuators configured to induce functional motion of one or more organs at least partially contained within the given system to mimic physiological organ movements (e.g., the motion of organs caused by a patient breathing, pain responses, or the like) during a medical procedure. For example, by causing the at least one organ to move in a manner mimicking physiological organ movements, the systems described herein may provide a more clinically relevant models of the at least one organ for use in training clinicians, developing new devices and/or therapies, or the like.

In some implementations, the system is configured to simulate actions of one or more nerves associated with the one or more organs. For example, the system may be configured to enable connection of one or more electrical devices for stimulating and/or recording activities with the nerves associated with the one or more organs and/or associated tissues. Additionally or alternatively, the system may be configured to simulate intrinsic actions and or the effects of therapies on one or more nerves.

The system also may optionally be configured to mimic vasculature access to the one or more organs. For example, for a system that houses at least one kidney, the associated renal vessels and renal nerves, and a portion of an aorta, the container may include access points that allow connection of the aorta to one or more tubes to simulate radial access, femoral access, brachial access, or the like.

In some examples, the system may be configured to simulate flow of other fluids to or within the one or more organs. For example, the system may include separate fluid circuits configured to mimic fluid flow through the kidney(s) and ureter.

The system may be used to investigate various aspects of various therapies, such as stenting, neuromodulation, ablation, denervation (e.g., renal denervation), or the like. In some implementations, the system may include or be usable with one or more sensors for sensing physiological parameters associated with the one or more organs. The one or more sensors may include, for example, electrical sensors (e.g., for sensing nerve action), one or more flow sensors (e.g., for sensing perfusate flow), one or more pressure sensors (e.g., for sensing perfusate pressure), one or more imaging devices, one or more thermocouples, one or more ultrasound sensors, or the like. The one or more sensors may be used to sense effects of therapies performed on the one or more organs.

Thus, systems described herein enable the mimicking, maintenance, and measurement of physiological conditions, such as physiological organ movements (e.g., caused by breathing), during perfusion of one or more organs. By mimicking the organ movement patterns, the model may yield more clinically relevant data and clinician training, potentially reducing the cost and length of pre-clinical and clinical testing and training and accelerating the development and deployment of effective medical devices and therapies.

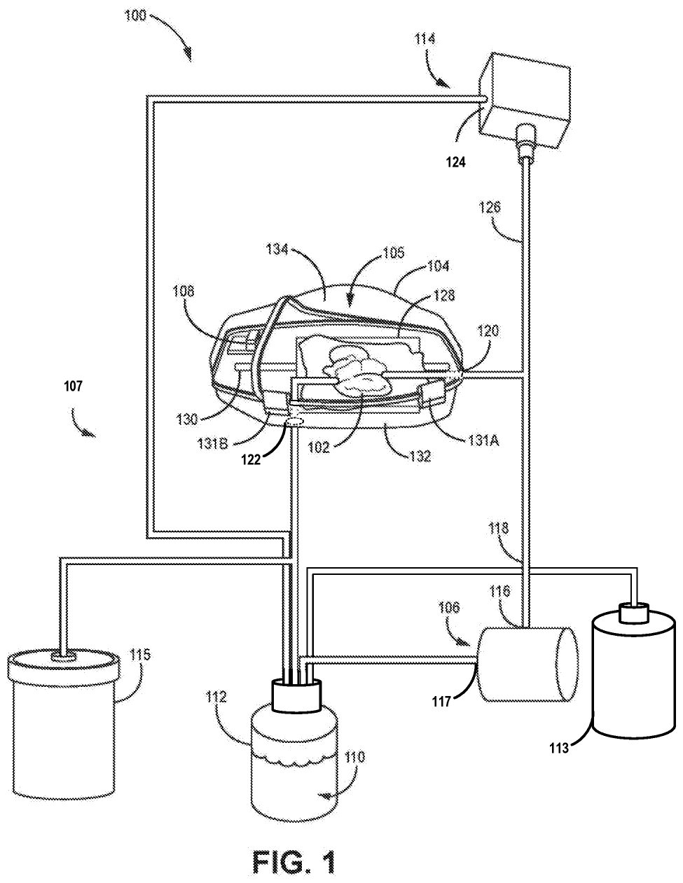

FIG. 1 is a conceptual diagram illustrating an example system 100 configured to induce one or more isolated organs 102 (“organs 102”) to mimic at least one physiologic condition. As used herein, organs 102 refers to organs and/or tissues (e.g., isolated tissues or tissues associated with one or more organs). System 100 includes an container 104 in which organs 102 are at least partially contained. For example, container 104 may define an interior cavity 105 configured to receive organs 102. Organs 102 may include one or more of kidneys, spleens, livers, etc. Organs 102 may be normal or pathologic. In some examples, container 104 may fully enclose organs 102, and in other examples, container 104 may only partially enclose organs 102. In any case, container 104 may represent a controlled environment, such as, e.g., a container containing fluid in which organs 102 are at least partially submerged.

System 100 also may include a pump 106 and at least one actuator 108. As described in greater detail below, system 100 may be configured to perfuse organ 102 with blood or a blood substitute via pump 106. At least one actuator 108 configured to induce motion of organ 102 to mimic physiological organ movements, such as motion of organs caused by a patient breathing, pain responses, or the like during a medical procedure. A controller or computing device may be configured to control the operation of actuator 108. For example, the controller or computing device may cause actuator 108 to move organs 102 at a specific speed at specific times and in specific directions. Thus, system 100 may enable organs 102 to represent a clinically relevant model for device and/or therapy investigations, training, and/or refinements in a potentially timely and cost-effective manner.

Container 104 may define an interior cavity 105 configured to receive organs 102. Interior cavity 105, and in turn container 104, may have any dimension suitable for enclosing one or more organs 102. The size and shape of container 104 may depend on the organs 102 which container 104 is configured to house. For example, as shown in FIG. 1, container 104 may be configured to house a left kidney, a right kidney, a portion of an aorta, and at least some surrounding tissue, e.g., including vasculature, nerves, or the like. As other examples, container 104 may be configured to house other organs, such as a liver, a spleen, a heart, one or two lungs, or the like. In some examples, container 104 may be configured to house large assemblages of organs, such as a larger, isolated portion of a mammal, such as a torso of a human including heart, lungs, kidneys, vasculature, nerves, and/or surrounding tissue.

As examples, container 104 may have a generally triangular, rectangular, circular, oval, or the like, cross-section. In some cases, cross sections of container 104 may vary along a length of container 104, such that a width, height, perimeter, and other geometric properties of container 104 are not necessarily uniform. For instance, as shown in FIG. 1, container 104 has a generally oval cross-section, but the oval cross-section varies in size and is smallest near the ends of container 104 and largest in the middle of container 104.

Container 104 may be configured to open and close. For example, container 104 may include one or more latches (e.g., latches 131A and 131B shown in FIG. 1 (collectively, “latches 131”)). Latches 131 may be operated to secure a first component 132 to a second component 134 of container 104. That is, latches 131 may be deployed to press first component 132 and second component 134 together to fix first component 132 to second component 134 and seal interior cavity 105. Conversely, latches 131 may be released to permit movement of first component 132 relative to second component 134 and access to interior cavity 105. In addition, container 104 may include one or more hinges to which first component 132 and second component 134 are rotatably attached. In this way, second component 134 may rotate relative to first component 132 in a first direction to open container 104, and second component 134 may rotate relative to first component 132 in a second direction to close container 104. In other examples, container 104 may include any other suitable configuration for housing one or more organs 102 and providing access to interior cavity 105, e.g., a housing with one or more doors, hatches, or the like.

As shown in FIG. 1, in some examples, container 104 may be substantially optically transparent. In other examples, container 104 may include one or more substantially optically transparent portions, e.g., in combination with one or more optically opaque portions. The substantially optically transparent portions or substantially optically transparent container 104 may facilitate viewing of organs 102 and other items within interior cavity 105.

Container 104 may be configured to substantially maintain at least one environmental condition associated with organs 102 within interior cavity 105. That is, container 104 may control, influence, or otherwise regulate one or more properties within interior cavity 105 affecting organs 102. As an example, container 104 may be configured to provide at least some thermal insulation to help maintain a temperature within interior cavity 105 at a normothermic temperature (e.g., between about 97° Farehnheit (F) and about 99° F.) to, for example, help mimic the environment of a thoracic cavity. In some examples, container 104 is configured to reduce heat transfer between interior cavity 105 and an environment external to container 104. For example, container 104 may be formed from or otherwise include (as, e.g., a layer, a coating, etc.) a material with relatively low thermal conductivity, such as a plastic.

In some examples, system 100 may include a heating element (not shown), such as a heating lamp, a heating pad, etc. configured to heat interior cavity 105. For instance, the heating element may heat interior cavity 105 to about a normothermic temperature, such as about 99° F. In some cases, the heating element may be controlled by a controller based on a signal from a temperature sensor within container 104, e.g., in a closed loop manner to maintain a temperature within container 104 within a predetermined range, e.g., at a normothermic temperature. By maintaining these environmental conditions, container 104 may, for example, facilitate collection of measurements from system 100 that are clinically relevant.

As shown in FIG. 1, system 100 may include a fluid circuit 107 configured to deliver a fluid 110, such as a perfusate, to organs 102 inside container 104. Fluid 110 may be any suitable fluid, though in some cases, fluid 110 is transparent to facilitate visualization and/or imaging. Examples of fluid 110 include water, blood, diluted blood, a Krebs-Henseleit buffer (e.g., a solution comprising sodium, potassium, chloride, calcium, magnesium sulfate, bicarbonate, phosphate, and glucose), or the like. In some examples, fluid 110 is oxygenated so that fluid 110 can transfer oxygen to organs 102. In some examples, fluid 110 includes additives, such as clinically delivered drugs, biomarkers (e.g., creatinine), or the like, that may modify the function of organs 102. The effect of such additives on organs 102 may be measured or otherwise evaluated using the techniques of this disclosure.

Fluid circuit 107 includes pump 106 configured to establish fluid communication with organs 102 and tubing or conduit configured to fluidly connect organs 102 and pump 106. Pump 106 may be configured to generate a pulsatile (e.g., of or marked by a periodically recurring alternate increase and decrease of a pressure or quantity) flow of fluid. Additionally or alternatively, pump 106 may be configured to generate a continuous flow of fluid. In some, but not all examples, fluid circuit 107 further includes a reservoir 112 and a pressure column 114. In addition, in some, but not all examples, fluid circuit 107 includes at least one additional reservoir 113. Reservoir 113 may be a separate reservoir from reservoir 112. As discussed in further detail below, reservoir 113 and reservoir 112 may be in fluid communication such that fluid in reservoir 113 may flow into reservoir 112.

Pump 106 may include a pump outlet 116 to which a pump inlet line 118 may be configured to fluidly couple. Pump 106 may be in fluid communication with organ 102 via pump inlet line 118. For example, container 104 may include an inlet 120 configured to allow passage of pump inlet line 118 into interior cavity 105. In some instances, pump inlet line 118 is fluidly coupled to organ 102, thereby establishing fluid communication between pump 106 and organ 102. As an example, pump inlet line 118 may be inserted into an access point (e.g., an artery, a femoral access, a radial access, etc.) of organ 102.

Pump 106 is configured to generate a flow of fluid 110 throughout fluid circuit 107 to enable perfusion. In some examples, one or more parameters of pump 106 may be configurable or controllable. Example parameters of pump 106 that may be controlled or configured may include an output phase (or pulse) ratio, output phase (or pulse) rate, or stroke volume. In this way, pump 106 may be configurable or controllable to mimic a range of pulse rates (heart rates), stroke volumes, or the like. For instance, a low relatively output phase ratio may be suitable for simulating a physiologic pressure waveform.

The pressure applied to fluid 110 by pump 106 may cause fluid 110 to perfuse throughout organs 102. For example, fluid 110 may circulate through and eventually drain from vessels of organs 102. In some examples, such as when organs 102 include one or more kidneys, organs 102 may include one or more ureters to enable collection and monitoring of urine output and composition. At least a portion of fluid 110 may pass through organs 102 and exit container 104. For instance, the portion of fluid 110 may flow into a drain 122 (e.g., an outlet) configured to receive fluid 110 from within interior cavity 105. In some cases, drain 122 may be in fluid communication with reservoir 112, which may be configured to store a first portion of fluid 110 in fluid circuit 107 to allow for recirculation of the first portion of fluid 110 in fluid circuit 107. In examples, pump 106 includes a pump inlet 117 configured to be in fluid communication with reservoir 112. In this way, drain 122 may deliver fluid 110 (including the first portion of fluid 110) to pump inlet 117 for circulation and recirculation of fluid 110 in fluid circuit 107. Additionally, or alternatively, drain 122 may be in fluid communication with a drain reservoir 115, which may be configured to store a second portion of fluid 110. Drain reservoir 115 may not be in fluid communication with pump inlet 117 such that the second portion of fluid 110 is not recirculated in fluid circuit 107.

As noted above, fluid in reservoir 113 (when present) is configured to flow into reservoir 112. For example, reservoir 113 may store a more fresh fluid (e.g., buffer) that enables the fluid of a different recirculating source to be changed by adding in the less used buffer while simultaneously draining the previously circulated fluid. The amount of fluid in reservoir 113 that flows into reservoir 112 may replace at least some of the fluid that drains into drain reservoir 115. In this way, system 100 may “change” the fluid (e.g., buffer) of reservoir 112 by adding “fresh” fluid while simultaneously draining used, circulated fluid.

The pressure of fluid 110 within system 100 may be adjustable. In some cases, fluid circuit 107 includes pressure column 114 configured to adjust the pressure of fluid 110 within system 100. Pressure column 114 may be a volume of fluid 110 disposed in a vessel 124 positioned at an elevation higher than that of organs 102. Pressure column 114 may be in fluid communication with pump 106 via pump inlet line 118, and pressure column 114 may be in fluid communication with organs 102 via a column inlet line 126. Column inlet line 126 may be fluidly coupled to organs 102 (e.g., column inlet line 126 may be inserted into an access point of organ 102).

FIG. 1 shows an exemplary configuration of fluid circuit 107. In the example of FIG. 1, pump 106 delivers fluid from reservoir 112 to organs 102 or pressure column 114. A portion of pump inlet line 118 in fluid communication with container 104 may be clamped such that, while this portion of pump inlet line 118 is clamped, pump 106 delivers fluid 110 to pressure column 114 but not container 104. Once a fluid level of pressure column 114 reaches a threshold level, fluid 110 in pressure column 114 may flow through an overflow line in fluid communication with reservoir 112. In some examples, once the flow of fluid 110 through the overflow line is constant, the portion of pump inlet line 118 may be unclamped to allow flow of fluid 110 into organs 102. All fluid 110 that exits organs 102 may drain from cavity 105 via drain 122. Drained fluid 110 may flow into one of reservoir 112 or drain reservoir 115 via a respective line of fluid circuit 107. It should be understood that other fluid circuit configurations are possible and that the examples provided herein are not intended to be limiting.

System 100 may be configured to move organs 102 to mimic physiological movement associated with organs 102, such as breathing or movement due to a pain response. For example, system 100 may include one or more actuators 108 (“actuator 108”) configured to move organs 102 relative to another portion of system 100. For instance, in the example shown in FIG. 1, actuator 108 is coupled to a platform 128 on which organs 102 rest. Actuator 108 is configured to move platform 128 relative to container 104. In other examples, actuator 108 may be configured to move container 104 relative to another reference point, e.g., a table or floor on which container 104 rests, such that organs 102 also move relative to the reference point. In any case, actuator 108 may move platform 128 (and thus organs 102) to mimic physiological movements (e.g., breathing) associated with organs 102, including aspects or the physiological movements, such as breathing rates, breathing depths, pain response movements, etc.

Actuator 108 may be configured to move organs 102 in one or more dimensions of a three-dimensional space (e.g., a Cartesian coordinate system). For example, actuator 108 may be configured to move organs 102 horizontally and/or vertically.

As shown in FIG. 1, platform 128 is positioned within interior cavity 105 of container 104. Container 104 may be configured to mechanically support platform 128 (e.g., container 104 may carry platform 128), and platform 128 may be configured to mechanically support organs 102 (e.g., platform 128 may carry organs 102). Platform 128 may be configured to move in response to force applied by actuator 108 (which may, e.g., be secured to an inner surface of container 104 defining interior cavity 105 and platform 128), thereby causing movement of organs 102. Organs 102 may be secured (e.g., using clamps, fasteners, etc.) to platform 128 to reduce or prevent movement of organ 102 relative to platform 128.

In another example, platform 128 may be positioned underneath container 104, and platform 128 may be configured to mechanically support container 104. As discussed above, platform 128 may be configured to move in response to force applied by actuator 108 (which may, e.g., be secured to an outer surface of container 104 and platform 128), thereby causing movement of container 104 and organs 102 within interior cavity 105. In this case, organs 102 may be secured to container 104 to reduce or prevent movement of organs 102 relative to container 104.

In some implementations, container 104 may include one or more structural features that define a path 130 to guide movement of platform 128. For example, as shown in FIG. 1, path 130 may be a linear railing extending from a first end of container 104 to a second end of container 104. Path 130 may mechanically communicate with platform 128 such that when actuator 108 induces movement of platform 128, the movement of platform 128 is constrained by path 130. For example, path 130 may be configured to allow movement of platform 128 in a direction along path 130 (e.g., parallel to path 130) but resist movement of platform 128 in any other direction (e.g., perpendicular to path 130). Path 130 may be formed from a material with a low friction coefficient to reduce resistance to movement of platform 128 in a direction along path 130, which may facilitate the mimicking of fine physiological movements associated with organ 102.

In this way, system 100, which includes actuator 108, may enable the mimicking, maintenance, and measurement of physiological conditions, such as physiological movements of organs 102 (e.g., caused by breathing or a pain response), during perfusion of organs 102. By mimicking the movement patterns of organs 102, system 100 may provide a more clinically relevant model that may yield more clinically relevant data and clinician training, potentially reducing the cost and length of pre-clinical and clinical testing and training and accelerating the development and deployment of effective medical devices and therapies.

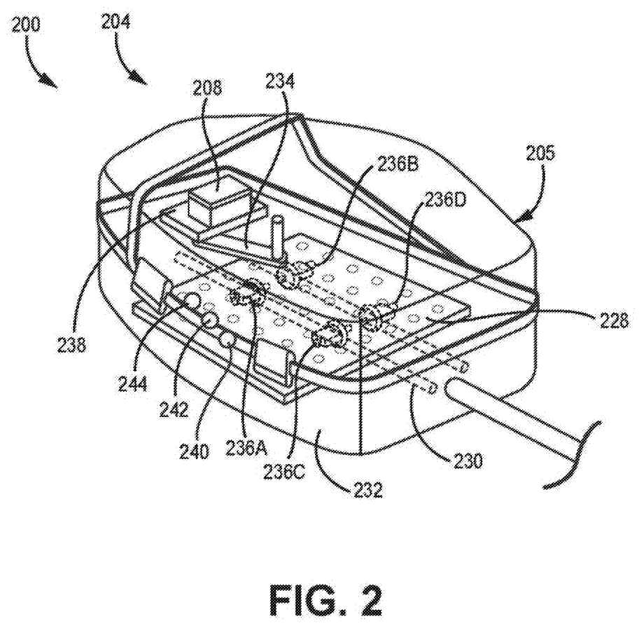

FIG. 2 is a conceptual diagram illustrating another example of a system 200 that includes an container 204 configured to induce organs 102 (FIG. 1) to mimic physiological conditions by moving organs 204. As shown in FIG. 2, container 204 includes a bottom portion 232 that defines a path 230. Path 230 is configured to mechanically guide movement of platform 228. In the illustrated example, platform 228 includes one or more rolling elements 236A-236D (collectively, “rolling elements 236”), such as wheels, disks, or the like, at least partially disposed within path 230. Accordingly, when actuator 208 applies force to platform 228, platform 228 may move substantially freely in accord with the applied force in a direction along path 230 but not in a perpendicular direction.

In some examples, actuator 208 may be mechanically supported by container 204. For example, actuator 208 may be attached to a support, such as a platform 238, of container 204. Actuator 208 may be configured to actuate an assembly arm 234 mechanically coupled to platform 228. For example, actuator 208 may be a servomotor that alternately rotates in a first direction and a second direction to induce corresponding movement of platform 238 in a direction along path 230, or may be a linear actuator.

As discussed above, container 204 may be configured to maintain one or more environmental conditions to, for example, facilitate measurements collected from system 200 that are clinically relevant. In examples, container 204 may be thermally insulated, in this way helping maintain temperature within interior cavity 205. Similarly, container 204 may be sealed (aside from designed inlets and outlets) to reduce or substantially prevent moisture from escaping interior cavity 205 aside from through the outlets. Alternatively, container 204 may include a vent or other aperture (not shown), the size of which may be adjustable in some cases, to allow vapor to flow out of interior cavity 205.

As shown in FIG. 2, system 200 may include one or more environmental sensors configured to measure one or more corresponding environmental conditions are disposed within interior cavity 205. For example, the environmental sensors may include a temperature sensor 240, a humidity sensor 242, a level sensor 244, or the like. Sensors 240, 242, and 244 may be separate or integrated into a sensor assembly. For example, temperature sensor 240, humidity sensor 242, and level sensor 244 may be integrated into a sensor assembly positioned within interior cavity 205.

Temperature sensor 240 may be configured to measure or sense the temperature of interior cavity 205. Temperature sensor 240 may be a contact sensor, a non-contact sensor, or the like. Examples of temperature sensor 240 include infrared (IR) sensors, thermocouples, thermistors, or the like.

Humidity sensor 242 may be configured to measure or sense the humidity in interior cavity 205. Humidity sensor 242 may be a capacitive humidity sensor, a resistive humidity sensor, a thermal conductivity humidity sensor, or the like.

Level sensor 244 may be configured to measure a fluid level of interior cavity 205 (e.g., an amount of fluid 110 (FIG. 1) in interior cavity 205). Level sensor 244 may be a point level sensor, a continuous level sensor, etc. Examples of level sensor 244 include a float switch, a photoelectric proximity sensor, a capacitance level sensor, a tuning fork sensor, ultrasonic level sensor, or the like.

In some examples, system 200 may include a component, such as a controller or computing device, that is configured to receive measurements from the environmental sensors (e.g., temperature sensor 240, humidity sensor 242, level sensor 244, or the like) and other sensors (e.g., pressure sensors) and monitor and/or record the measurements. In some implementations, the controller or computing device may be configured to control environmental conditions of interior cavity 105, operation of one or more actuator, and/or other parameters (e.g., pre-load pressure and/or after-load pressure of fluid 110) of system 200 based on the measurements received from sensors 240, 242, 244, and/or other sensors. In some implementations, the controller or computing device may implement a closed loop feedback control system. That is, the controller or computing device may receive one or more set points associated with operation of one or more components system 200 and substantially maintain the operation of the one or more components at or near the one or more set points by controlling actuators (e.g., actuator 108), heating elements, and/or other components of system 200.

For example, the controller or computing device may be configured to control operation of actuator 108 (FIG. 1) to mimic functional physiological movement of organs 102 (FIG. 1). For instance, the controller or computing device may be configured to control a movement amount (e.g., amplitude) and/or rate (e.g., frequency) of actuator 108 to mimic continuous functional physiological movements (e.g., breathing), control a movement amount (e.g., amplitude), duration, and/or speed of periodic or non-repetitive functional physiological movements (e.g., pain responses), or the like. A user may input values for one or more of these parameters to the controller or computing device, the controller or computing device may include predetermined values for one or more of these parameters, or combinations thereof.

For instance, to enable actuator 108 to mimic motion of organs 102 due to respiration, the controller or computing device may be configured with a movement amount (e.g., to mimic an amount of movement of organs 102 in or more directions due to a breath) and a movement frequency (e.g., to mimic a respiration rate). The controller or computing device may control actuator 108 to move platform 228 the configured amount at the configured rate to cause organs 102 (FIG. 1) to move and mimic physiological functional movement of organs 102).

As another example, system 200 may be configured to control a temperature within interior cavity 205 of container 204. A temperature set point may include single value or range of values at or around a normothermic temperature (e.g., a single value of 37° C. or a temperature range of 36° C. to 38° C.). The controller or computing device may be configured to control one or more heating elements that heat interior cavity 205 to substantially maintain the temperature of interior cavity 205 (as sensed by temperature sensor 240) at or near the single value set point or within the setpoint range. For instance, if the controller or computing device determines that a temperature within interior cavity 205 is below a normothermic range (e.g., less than about 36° C.) based on measurements from temperature sensor 240, the controller or computing device may control a heating element of system 200 to increase a temperature of fluid 110 (which is in fluid communication with interior cavity 205) or control a heating element that directly heats interior cavity 205 to output more heat until the temperature within interior cavity 205 is within the normothermic range. Conversely, if the controller or computing device determines that a temperature within interior cavity 205 is above a normothermic range (e.g., greater than about 38° C.) based on measurements from temperature sensor 240, the controller or computing device may control a heating element of system 200 to decrease a temperature of fluid 110 (which is in fluid communication with interior cavity 205) or control a heating element that directly heats interior cavity 205 to output less heat until the temperature within interior cavity 205 is not above the normothermic range but instead within the normothermic range. The controller or computing device may implement the temperature control using any suitable control scheme, such as proportional control, integral control, derivative control, proportional and integral control, proportional and derivative control, proportional integral derivative control, or the like.

As another example, system 200 may be configured to control a humidity level within interior cavity 205 of container 204. A humidity set point may include single humidity value or range of humidity values. The controller or computing device may be configured to control a fluid source, a wet gas source, or the like to control humidity within interior cavity 205. For instance, if the controller or computing device determines that a humidity within interior cavity 205 is below a pre-determined humidity range based on measurements from humidity sensor 242, the controller or computing device may output an indication that a size of a vent of container 204 should be decreased, directly control an actuator to close the vent to decrease outflow of vapor, and/or control an air input that passes through a humidifier to increase the inflow of (humid) air and/or increase the humidity of the air until the humidity within interior cavity 205 is within the pre-determined humidity range. Conversely, if the controller or computing device determines that a humidity within interior cavity 205 is above a pre-determined humidity range based on measurements from humidity sensor 242, the controller or computing device may output an indication that a size of a vent of container 204 should be increased, directly control an actuator to open the vent to increase outflow of vapor, and/or control an air input that passes through a humidifier to decrease the inflow of (humid) air and/or decrease the humidity of the air until the humidity within interior cavity 205 is no longer above the pre-determined humidity range but instead within the pre-determined humidity range. The controller or computing device may implement the humidity control using any suitable control scheme, such as proportional control, integral control, derivative control, proportional and integral control, proportional and derivative control, proportional integral derivative control, or the like.

As yet another example, system 200 may be configured to control a fluid level within interior cavity 205 of container 204. A fluid level set point may include single fluid level value or range of fluid level values. The controller or computing device may be configured to control a valve, pump, or the like to control the fluid level within interior cavity 205. if the controller or computing device determines that a fluid level within interior cavity 205 is below a pre-determined fluid level range based on measurements from level sensor 244, the controller or computing device may control a valve to close and decrease a flow rate through drain 122 (FIG. 1) until the fluid level is within the pre-determined fluid level range. Conversely, if the controller or computing device determines that a fluid level within interior cavity 205 is above a pre-determined range based on measurements from level sensor 244, the controller or computing device may control a valve to open further to increase a flow rate through drain 122 until the fluid level is within the pre-determined fluid level range. The controller or computing device may implement the fluid level control using any suitable control scheme, such as proportional control, integral control, derivative control, proportional and integral control, proportional and derivative control, proportional integral derivative control, or the like.

As yet another example, system 200 may be configured to control a pre-load pressure of fluid flowing through fluid circuit 107 (FIG. 1) into vasculature associated with organs 102. The pre-load pressure refers to pressure of fluid within fluid circuit 107 prior to organs 102. The controller or computing device may receive a pre-load pressure set point, which may indicate a desired pre-load pressure. If the controller or computing device determines that a measures pre-load pressure is below a pre-load pressure set point based on measurements from at least one pressure sensor disposed within fluid circuit 107 prior to organs 102, the controller or computing device may cause an actuator operably coupled to pressure column 114 to increase the height of pressure column 114 until the pre-load pressure is substantially the same as the pre-load pressure set point. Conversely, if the controller or computing device determines that a pre-load pressure is above the pre-load pressure set point based on measurements from at least one pressure sensor disposed within fluid circuit 107 prior to organs 102, the controller or computing device may cause the actuator operably coupled to pressure column 114 to decrease the height of pressure column 114 until the pre-load pressure is substantially the same as the pre-load pressure set point. The controller or computing device may implement the pre-load pressure control using any suitable control scheme, such as proportional control, integral control, derivative control, proportional and integral control, proportional and derivative control, proportional integral derivative control, or the like. The controller may similarly control a valve to control an after-load pressure (e.g., a pressure of fluid within fluid circuit 107 downstream of organs 102).

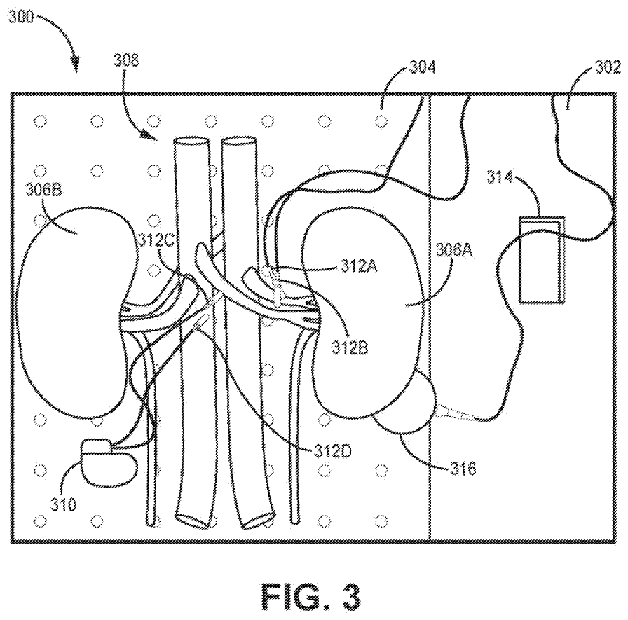

In some examples, a system may be configured to mimic additional or alternative physiologic conditions. For example, a system may be configured to mimic nervous system action to facilitate testing or training on techniques that modulate nerve activity, such as renal denervation techniques. FIG. 3 is a conceptual diagram illustrating a system 300 that includes an container 302 and a platform 304 supporting two kidneys, renal vasculature, and surrounding tissue, including a portion of an aorta and renal nerves. As shown in FIG. 3, kidneys 306A and 306B (collectively, “kidneys 306”) and other tissues 308 are positioned on platform 304. System 300 may be configured to stimulate nerves (e.g., to simulate the operation of the nerves), record nerve compound action potentials (e.g., spontaneous and induced), or both.

Container 304 may be configured to allow a variety of equipment, such as a medical device 310, one or more electrodes 312A-312D (collectively, “electrodes 312”), or the like, to be positioned within interior cavity 105 (FIG. 1). For example, container may include an access port 314 configured to allow insertion of equipment, such as medical device 310, electrodes 312, into interior cavity 105 to access the at least one organ (such as kidneys 306, tissue 308, or the like). Access port 314 may be positioned on any portion of container 304, such as a sidewall of container 304.

Medical device 310 may be configured to monitor or treat conditions or functions relating to the at least one organ. For example, medical device 310 may be an electrical stimulation device, a drug delivery device, a sensing device, a catheter, or the like. Medical device 310 may be configured to electrically stimulate one or more nerves to modulate activity of the one or more nerves. The one or more nerves may include a portion of the central nervous system or a portion of the peripheral nervous system. The one or more nerves may include sensory or motor nerves, nerves of the somatic or autonomic systems, or sympathetic or parasympathetic nerves.

In some examples, medical device 310 may be configured to ablate nervous tissue, e.g., using a pulsed electric field, RF energy, microwave energy, a chemical agent, ultrasound energy, heat, cold, or the like.

As another example, medical device 310 may be configured to deliver a second medical device to a location within the at least one organ. For instance, medical device 310 may include a delivery catheter, a balloon catheter, or the like configured to deliver a stent, a drug delivery implant, a replacement valve, or the like to a target location within the at least one organ.

As another example, medical device 310 may be configured to sense one or more physiological parameters associated with the at least one organ. For example, medical device 310 may include an electrode configured to sense electrical signals associated with the at least one organ, a pressure sensor configured to sense a pressure of the fluid flowing through the at least one organ, a flow sensor configured to sense a flow rate of the fluid flowing through the at least one organ, an imaging device configured to image a portion of the at least one organ, or the like. For example, medical device 310 may include an endoscopic camera, thermocouple, a catheter-carried ultrasound sensor, or the like. In some examples, medical device 310 is implantable within the at least one organ. In other examples, medical device 310 may include a portion that is configured to be introduced (e.g., intravascularly) to the at least one organ and a portion that is configured remain external to the at least one organ (e.g., external to the patient).

Medical device 310 may be the portion of the system that is being developed or on which the clinician is being trained. System 300 may include one or more access sites configured to mimic access to the at least one organ via a standard approach, such as radial artery access, femoral artery access, brachial artery access, or the like. For example, in system 300, which include part of an aorta and two kidneys 306, a fluid inlet tube may be attached to one end of the aorta (mimicking inflow of blood into the portion of the aorta as would occur in an intact organ system) and a fluid outlet tube may be attached to the other end of the aorta (mimicking outflow of blood from the portion of the aorta as would occur in an intact organ system). The fluid inlet tube may be shaped, sized, and positioned to approximate a typical aorta extending superior to the kidneys and include an access port or branch approximating the brachial artery and/or radial artery. Additionally, or alternatively, the fluid outlet tube may be shaped, sized, and positioned to approximate a typical aorta extending inferior to the kidneys and include an access port or branch approximating a femoral artery. In this way, system 300 may allow approximation or mimicking of femoral, brachial, and/or radial access to the at least one organ.

Electrodes 312 may be positioned at one or more target locations of the at least one organ (such as kidneys 306, tissue 308, or the like) for exciting nerves associated with the at least one organ to mimic or simulate nervous tissue function (e.g., action potentials). For example, electrodes 148 may be implanted at target locations; attached to one or more nerves using alligator clips, needles or another suitable electrical connection; and driven by a stimulator configured to imitate or simulate nervous tissue function. In this way, system 300 may approximate or mimic function of nerves associated with the at least one organ, which may allow evaluation of therapies configured to affect nerve tissue or nervous system function (such as neuromodulation, denervation (or nerve ablation), or the like.

System 100 may include one or more physiological sensors 316 configured to measure at least one physiological parameter (e.g., pulse rate, a pulse depth, a temperature, a nerve activity, or the like) associated with the at least one organ (e.g., kidney 306A). Examples of physiological sensors 152 may include an electrode, a flow transducer, a pressure sensor, a temperature sensor such as a thermocouple, etc. In some cases, therapy delivery may induce changes (which may be desired or unintended consequences of the therapy) in physiological parameters, such as nerve activity, which system 300 may monitor (e.g., via physiological sensors 316), quantify, and compare. In this way, system 300 may facilitate the creation and testing of medical 310 and therapies delivered using medical device 310. Similarly, system 300 may be configured to monitor, quantify, and compare organ secretions before, during, and after therapy delivery. For example, if the at least one organ in container 304 is a kidney 306A or 306B, a physician or other user of system 300 may cannulate the ureters, and system 300 may collect and monitor urine output and composition.

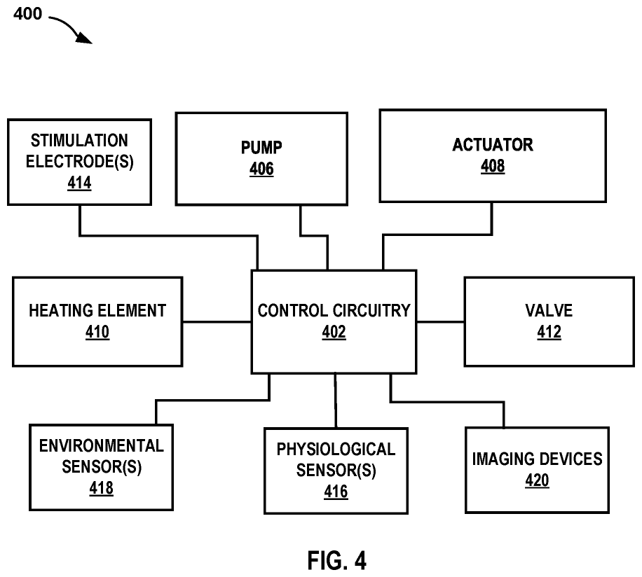

In some examples, systems 100, 200, and 300 are at least partially manually operated. Systems 100, 200, and 300 also may be partially or fully automated. FIG. 4 is a conceptual diagram of example control circuitry 402 configured to control components of a system 400. Control circuitry 402 may include processing circuitry (e.g., one or more processors or logic circuits) configured to control one or more components of system 400, such as pump 406, actuator 408, a heating element 410 configured to heat fluid 110 and/or container 104 (FIG. 1), a valve 412 configured to regulate flow rate through drain 122 (FIG. 1), one or more stimulation electrodes 414, one or more physiological sensors 416, one or more environmental sensor 418, one or more imaging devices 420, or the like. Control circuitry 402 may implement any of the controls described above with reference to FIG. 2. For example, control circuitry 402 may control pump 406 to regulate a pressure and flow rate of the flow of fluid 110 (FIG. 1), an elevation of fluid 110 in pressure column 114 (FIG. 1; in this way adjusting the pressure of fluid 110 prior to organs 102), or the like. Control circuitry 402 may also control actuator 408 to move platform 128 to cause at least one organ 102 (FIG. 1) to move in a predetermined manner. For example, control circuitry 402 may control actuator 408 to induce platform 128 to move back and forth along path 130 at a specific speed, frequency, distance, and the like, as described above.

In some examples, control circuitry 402 receives signals from one or more one or more environmental sensors 418 (which may correspond to, e.g., temperature sensor 240, humidity sensor 242, and level sensor 244 of FIG. 2) and controls components of system 400 based on the signals to regulate environmental conditions for obtaining clinically relevant data from at least organ 102, maintaining viability of organ 102, and the like. For example, if control circuitry 402 receives sensor data from temperature sensor 240 indicating that the temperature within interior cavity 205 is below a normothermic temperature range, control circuitry 402 may control heating element 410 to increase the temperature of fluid 110 and/or to heat interior cavity 205 until the sensed temperature is within the normothermic temperature range.

One or more techniques described as being performed by control circuitry 402 may at least partially be performed by another component or manually by a user. For example, instead of control circuitry 402 controlling components of system 400 to regulate environmental conditions, a user (e.g., a researcher or clinician) of system 400 may manually adjust a height of pressure column 114, a power of heating element 410, a position of valve 412, etc., to control a pressure of fluid 110, a temperature of fluid 110 and/or interior cavity 105, a flow rate of fluid 110, or the like.

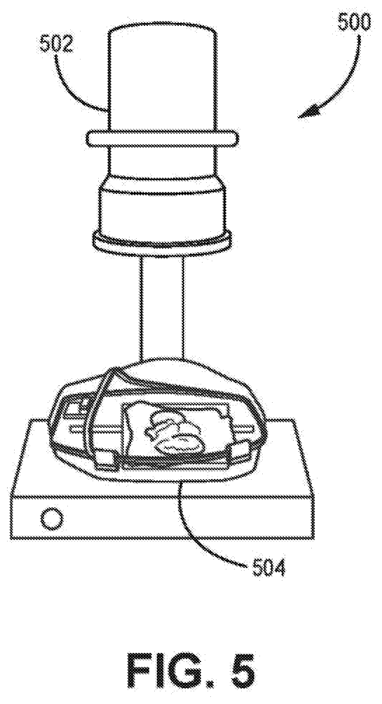

FIG. 5 is a conceptual diagram of an example system 500 configured to enable visualization of at least one organ 102. System 500 may be used with any of systems 100, 200, 300, or 400. In some examples, fluid 110 may be transparent to facilitate visualization by one or more image sensors 502, such as an external camera (as shown in FIG. 5, which may be an overhead camera), an endoscopic camera, an imaging system, a fluoroscopic imaging device (e.g., fluoroscopic imaging with a C-arm fluoroscopy machine), a thermal imaging device (e.g., an external thermal camera), an optical coherence tomography device, a IBIS device, an intravascular ultrasound device, a 4D echo device, an external ultrasound device, or the like.

In some examples, system 500 may be used to measure histopathology. For instance, a clinician may perform post-procedural histopathology on organ 102 by staining organ 102 while organ 102 is positioned in a container 504 and visually inspect organ 102 using image sensors 502 to see the extent of staining. In this way, system 500 may increase accessibility to organ 102 during testing. Alternatively, the clinician may remove organ 102 from container 504 and perform standard histopathology, reposition organ 102 in container 504, and visually inspect organ 102 using image sensors 502 to see the extent of staining.

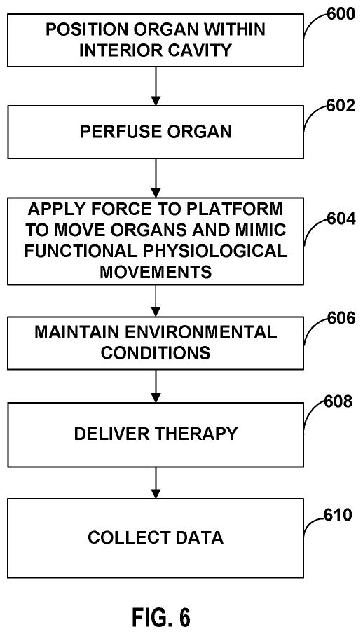

FIG. 6 is a flow diagram that illustrates an example technique for operating system 100. Although described with reference to FIG. 1, it will be appreciated that a similar technique may be performed by system 200, system 300, or system 400, alone or in combination with system 500. Further, the systems described herein may perform other techniques. Although FIG. 6 illustrates a sequence of steps, one or more of the steps may be performed in a different order and/or concurrently with one or more other steps. As shown in FIG. 6, at least one organ 102 may be positioned within interior cavity 105 (600). In some examples, latches 131 are released to permit first component 132 to separate from second component 134, permitting access to interior cavity 105. At least one organ 102 may be inserted into interior cavity 105 and place on, for example, platform 128. Latches 131 may then be actuated to press first component 132 and second component 134 together to seal interior cavity 105.

Pump 106 may be fluidly coupled to at least organ 102 to perfuse at least one organ 102 (602). As an example, pump inlet line 118 may be inserted into an access point of organ 102. For instance, pump inlet line 118 or column inlet line 126 may be coupled to a superior portion of the aorta shown in FIGS. 1 and 3. An outlet line may be coupled to an inferior portion of the aorta shown in FIGS. 1 and 3 and drain to drain reservoir 112 and/or reservoir 115. Pump 106 may generate a flow of fluid 110 where the pressure of fluid 110 causes fluid 110 to perfuse throughout organ 102. At least a portion of fluid 110 may pass through vasculature associated with at least one organ 102 and exit container 104. For instance, the portion of fluid 110 may flow out of drain 122 (e.g., via the outlet line) that receives fluid 110 from within interior cavity 105. In some cases, drain 122 may be in fluid communication with reservoir 112, which may store fluid 110 in fluid circuit 107. In some examples, pump 106 includes pump inlet 117 configured to be in fluid communication with reservoir 112. In this way, drain 122 may deliver at least a portion of fluid 110 to pump inlet 117 for recirculation of fluid in fluid circuit 107.

Actuator 108 may apply force to platform 128, thereby moving organ 102 to mimic physiological movements of at least one organ 102 (604). In examples, platform 128 may move substantially freely in a direction along path 130 but not in other directions. Actuator 108 may be a servomotor that alternately rotates in a first direction and a second direction to induce corresponding movement of platform 138 in a direction along path 130, a linear actuator, or the like.

Container 104 may maintain one or more environmental conditions to, for example, facilitate clinically relevant measurements collected from system 100 (606). In some examples, container 104 provides thermal insulation to help maintain a desired temperature within interior cavity 105. Similarly, container 104 may be sealed to prevent humidity from escaping interior cavity 105. In some cases, system 100 may include control circuitry (e.g., control circuitry 402 shown in FIG. 4) that controls one or more components of system 100, such as pump 406, actuator 408, heating element 410, valve 412, or the like to help maintain one or more environmental conditions within container 104. For example, control circuitry 402 may control pump 406 to regulate a pressure of the flow of fluid 110, an elevation of fluid 110 in pressure column 114 (in this way adjusting the pressure of fluid 110), or the like. Control circuitry 402 may also control actuator 408 to move platform 128 to cause organ 102 to move in a predetermined manner. For example, control circuitry 402 may control actuator 108 to induce platform 128 to move back and forth along path 130 at a specific speed, frequency, distance, and the like.

In some examples, control circuitry 402 receives electrical signals from one or more environmental sensors 415 (which may correspond to, e.g., temperature sensor 240, humidity sensor 242, and level sensor 244) and controls components of system 100 to regulate environmental conditions to facilitate obtaining clinically relevant data from at least one organ 102, maintaining viability of at least one organ 102, or the like.

One or more medical devices (e.g., medical device 310) and/or one or more therapies may be tested using system 100 (608). For example, medical device 310 may be used to deliver a therapy or device to at least one organ 102, and effects of the delivery of the therapy or device may be sensed using physiological sensors 316, imaging devices 420 or 502, or the like.

Sensors (e.g., physiological sensors 416, image devices 420, or the like) may collect data from system 100 (610). For example, physiological sensors 416 may monitor nerve activities associated with organ 102 that are affected by delivery of therapy. In this way, system 100 may facilitate the creation and testing of devices (e.g., medical device 310) that can affect nerve action. Similarly, system 100 may be configured to monitor, quantify, and compare organ secretions before, during, and after therapy delivery. For example, if at least one organ 102 in the container is a kidney, a physician or other user of system 100 may cannulate the ureters, and system 100 may collect and monitor urine output as well as compositions (e.g., with a sample analyzer). In some examples, if at least one organ 102 is a kidney, flow rate in and out of organ 102 may be used to determine vascular resistance.

Although FIG. 6 illustrates a sequence of steps, one or more of the steps 602-610 may be performed in a different order and/or at least partially concurrently with one or more other steps. For example, although perfusing organ (602) is illustrated as the second step of FIG. 6, perfusing organ (602) may continue while steps 604-610 are performed, and may be initiated before, during, or after initiation of steps 604, 606, and/or 608. Similarly, although steps 608 and 610 are illustrated as sequential steps, steps 608 and 610 may be performed at least partially concurrently, step 610 may be initiated before 608, or the like. Other combinations or permutations of the order and duration of steps 600-610 will be apparent to a person having ordinary skill in the art and are within the scope of this disclosure.

As an example, perfusing organs (602) may be initiated before one or more of inducing physiological organ movement (604), maintaining environmental conditions of the container (606), delivering therapy (608), and collecting data (610), or perfusing organs (602) may be performed at least partially concurrently with one or more of inducing physiological (604), maintaining environmental conditions of the container (606), delivering therapy (608), or collecting data (610). As another example, perfusing organs (602) may be initiated after one or more of inducing physiological organ movement (604), maintaining environmental conditions of the container (606), delivering therapy (608), and collecting data (610), and perfusing organs (602) may be performed at least partially concurrently with one or more of inducing physiological (604), maintaining environmental conditions of the container (606), delivering therapy (608), or collecting data (610). As such, the order of any of steps 602-610 may be modified as appropriate (e.g., step 608 may be initiated before step 604, step 610 may be initiated before step 608, etc.). Similarly, the duration of any of steps 602-610 may be such that one or more of steps 602-610 are performed at least partially concurrently (e.g., step 602 may be performed concurrently with step 604, step 606, and step 610). In some implementations, steps 602-610 are all performed at least partially concurrently, though they may be initiated at various times and in a different order than illustrated in FIG. 6. Further, one or more of steps 602-610 may be repeated from time to time (e.g., periodically) while one or more other steps of steps 602-610 are being performed. For example, one or both of delivering therapy (608) or collecting data (610) may be performed from time to time (e.g., discontinuously) while perfusing organs (602), inducing physiological organ movement (604), and/or maintaining environmental conditions of the container (606).

This disclosure includes various examples, such as the following examples.

Example 1: A system includes a pump configured to generate a flow of a fluid; a container, defining an interior cavity, configured to receive at least one organ or tissue and maintain at least one environmental condition associated with the at least one organ or tissue within the interior cavity, a fluid circuit configured to fluidically couple the pump to the at least one organ or tissue; a platform operably coupled to the container; and at least one actuator configured to move the platform to mimic at least one biological movement associated with the at least one organ or tissue.

Example 2: The system of example 1, wherein the pump includes a pump outlet, wherein the fluid circuit includes an inlet line configured to fluidically couple to the pump outlet, and wherein the container includes an inlet configured to allow passage of the inlet line into the interior cavity of the container to fluidically couple to the at least one organ or tissue.

Example 3: The system of example 1 or example 2, wherein the container further includes a drain configured to: receive, from within the interior cavity, a portion of the fluid; and deliver the portion of the fluid to a pump inlet of the pump.

Example 4: The system of any one of examples 1 to 3, wherein the fluid circuit further includes a pressure column configured to adjust a pressure of the fluid within the at least one organ or tissue.

Example 5: The system of any one of examples 1 to 4, further including control circuitry configured to control the pump to regulate the flow of the fluid.

Example 6: The system of any one of examples 1 to 5, further including control circuitry configured to control the at least one actuator to move the platform.

Example 7: The system of any one of examples 1 to 6, wherein the at least one actuator includes a linear actuator.

Example 8: The system of any one of examples 1 to 7, wherein the at least one actuator includes a servomotor.

Example 9: The system of any one of examples 1 to 8, wherein the platform is positioned within the interior cavity of the container, wherein the platform is configured to mechanically support the at least one organ or tissue, and wherein the container is configured to mechanically support the platform.

Example 10: The system of example 9, wherein the container defines a path configured to guide movement of the platform.

Example 11: The system of example 10, wherein the path includes at least one of a groove or a railing.

Example 12: The system of any one of example 1 to 8, wherein the platform is positioned underneath the container, and the platform is configured to mechanically support the container, and wherein the container is configured to mechanically support the at least one organ or tissue.

Example 13: The system of any one of examples 1 to 12, wherein the container further includes an access port configured to allow insertion of a medical device into the interior cavity.

Example 14: The system of example 13, wherein the access port is positioned on the container to facilitate simulation of at least one of femoral access or radial access to the at least one organ or tissue.

Example 15: The system of example 13 or 14, wherein the medical device includes a lead carrying at least one electrode.

Example 16: The system of any one of examples 1 to 15, wherein the at least one environmental condition includes at least one of a temperature, a humidity, or a fluid level, wherein the fluid level is an amount of fluid in the interior cavity.

Example 17: The system of any one of examples 1 to 16, further including at least one environmental sensor configured to measure the at least one environmental condition.

Example 18: The system of example 17, wherein the at least one environmental sensor includes at least one of a temperature sensor, a humidity sensor, or a weight sensor.

Example 19: The system of any one of examples 1 to 18, wherein the fluid is transparent.

Example 20: The system of any one of examples 1 to 19, wherein the fluid includes a solution including sodium, potassium, chloride, calcium, magnesium sulfate, bicarbonate, phosphate, and glucose.

Example 21: The system of any one of examples 1 to 20, wherein the fluid includes blood.

Example 22: The system of example 21, wherein the fluid includes diluted blood.

Example 23: The system of any one of examples 1 to 22, wherein the container is configured to receive at least a portion of at least one of a kidney, a liver, a spleen, a heart, a lung, skeletal muscle tissue, an eye, or a gallbladder.

Example 24: The system of any one of examples 1 to 23, further including at least one physiological sensor configured to measure at least one physiological condition associated with the at least one organ or tissue.

Example 25: The system of example 24, wherein the at least one physiological condition includes at least one of a pulse rate, a pulse depth, a pressure of the fluid, a flow rate of the fluid, or a nerve activity.

Example 26: The system of example 24 or 25, wherein the at least one physiological sensor includes at least one of an electrode, a flow transducer, or a pressure sensor.

Example 27: The system of any one of examples 1 to 26, further including an electrode, positioned within the interior cavity, configured to deliver electrical signals to at least one nerve associated with the at least one organ or tissue.

Example 28: The system of any one of examples 1 to 27, further including at least one image sensor configured to collect image data of the at least one organ or tissue.

Example 29: The system of example 28, wherein the at least one image sensor includes at least one of an endoscopic camera, an external camera, or an imaging system positioned outside the container.

Example 30: A method of operating a perfusion system includes generating, by a pump, a flow of a fluid through a fluid circuit, wherein the pump includes a pump outlet, and wherein the fluid circuit is configured to fluidically couple the pump to at least one organ or tissue such that the fluid flows through the at least one organ or tissue in response to the pump generating the flow of the fluid, wherein the perfusion system includes a container defining an interior cavity configured to receive at least one organ or tissue, and wherein the container is configured to maintain at least one environmental condition associated with the at least one organ or tissue within the interior cavity; and moving, by at least one actuator operably coupled to a platform operably coupled to the container, the platform to mimic at least one biological movement associated with the at least one organ or tissue.

Example 31: The method of example 30, wherein the pump includes a pump outlet, wherein the fluid circuit includes an inlet line configured to fluidically couple to the pump outlet, and wherein the container includes an inlet configured to allow passage of the inlet line into the interior cavity of the container to fluidically couple to the at least one organ or tissue.

Example 32: The method of example 30, wherein the container further includes a drain configured to: receive, from within the interior cavity, a portion of the fluid; and deliver the portion of the fluid to a pump inlet of the pump.

Example 33: The method of any one of examples 30 to 32, wherein the fluid circuit further includes a pressure column, and wherein the method further includes: controlling a height of the pressure column to control a pressure of the fluid within the at least one organ or tissue.

Example 34: The method of any one of examples 30 to 33, wherein generating, by the pump, the flow of the fluid through the fluid circuit include: controlling, by control circuitry, the pump to generate and regulate the flow of the fluid.

Example 35: The method of any one of examples 30 to 34, wherein moving, by the actuator operably coupled to the platform operably coupled to the container, the platform to mimic at least one biological movement includes: controlling, by control circuitry, the at least one actuator to move the platform.

Example 36: The method of any one of examples 30 to 35, wherein the at least one actuator includes a linear actuator.

Example 37: The method of any one of examples 30 to 36, wherein the at least one actuator includes a servomotor.

Example 38: The method of any one of examples 30 to 37, wherein the platform is positioned within the interior cavity of the container, wherein the platform is configured to mechanically support the at least one organ or tissue, and wherein the container is configured to mechanically support the platform.

Example 39: The method of example 38, wherein the container defines a path configured to guide movement of the platform, and wherein moving, by the at least one actuator operably coupled to the platform operably coupled to the container, the platform to mimic at least one biological movement includes moving, by the at least one actuator operably coupled to the platform operably coupled to the container, the platform along the path.

Example 40: The method of example 39, wherein the path includes at least one of a groove or a railing.

Example 41: The method of any one of examples 30 to 37, wherein the platform is positioned underneath the container, and the platform is configured to mechanically support the container, and wherein the container is configured to mechanically support the at least one organ or tissue.

Example 42: The method of any one of examples 30 to 41, further including inserting a medical device into the interior cavity via an access port in the container.

Example 43: The method of example 42, wherein the access port is positioned on the container to facilitate simulation of at least one of femoral access or radial access to the at least one organ or tissue.

Example 44: The method of example 42 or 43, wherein the medical device includes a lead carrying at least one electrode.

Example 45: The method of any one of examples 30 to 44, wherein the at least one environmental condition includes at least one of a temperature, a humidity, or a fluid level, wherein the fluid level is an amount of fluid in the interior body cavity.

Example 46: The method of any one of examples 30 to 45, further including measuring the at least one environmental condition using at least one environmental sensor.

Example 47: The method of example 46, wherein the at least one environmental sensor includes at least one of a temperature sensor, a humidity sensor, or a weight sensor.

Example 48: The method of any one of examples 30 to 47, wherein the fluid is transparent.

Example 49: The method of any one of examples 30 to 48, wherein the fluid includes a solution including sodium, potassium, chloride, calcium, magnesium sulfate, bicarbonate, phosphate, and glucose.

Example 50: The method of any one of examples 30 to 49, wherein the fluid includes blood.

Example 51: The method of example 50, wherein the fluid includes diluted blood.

Example 52: The method of any one of examples 30 to 51, wherein the at least one organ or tissue includes at least a portion of at least one of a kidney, a liver, a spleen, a heart, a lung, skeletal muscle tissue, an eye, or a gallbladder.

Example 53: The method of any one of examples 30 to 52, further including measuring at least one physiological condition associated with the at least one organ or tissue using at least one physiological sensor.

Example 54: The method of example 53, wherein the at least one physiological condition includes at least one of a pulse rate, a pulse depth, a pressure of the fluid, a flow rate of the fluid, or a nerve activity.

Example 55: The method of example 53 or 54, wherein the at least one physiological sensor includes at least one of an electrode, a flow transducer, or a pressure sensor.

Example 56: The method of any one of examples 30 to 55, further including delivering electrical signals to at least one nerve associated with the at least one organ or tissue using an electrode positioned within the interior cavity of the container.

Example 57: The method of any one of examples 30 to 56, further including imaging the at least one organ or tissue using at least one image sensor.

Example 58: The method of example 57, wherein the at least one image sensor includes at least one of an endoscopic camera or an external camera positioned outside the container. in accordance with any of the techniques described in this disclosure.

The techniques described in this disclosure may be implemented, at least in part, in hardware, software, firmware or any combination thereof. For example, various aspects of the described techniques, including those described with respect to control circuitry 402, may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

What is claimed is:

1. A system comprising:
a pump configured to generate a flow of a fluid;
a container, defining an interior cavity, configured to receive at least one organ or tissue and maintain at least one environmental condition associated with the at least one organ or tissue within the interior cavity,
a fluid circuit configured to fluidically couple the pump to the at least one organ or tissue;
a platform operably coupled to the container; and
at least one actuator configured to move the platform to mimic at least one biological movement associated with the at least one organ or tissue.

2. The system of claim 1, wherein the pump comprises a pump outlet, wherein the fluid circuit comprises an inlet line configured to fluidically couple to the pump outlet, and wherein the container comprises an inlet configured to allow passage of the inlet line into the interior cavity of the container to fluidically couple to the at least one organ or tissue.

3. The system of claim 1, wherein the fluid circuit further comprises a pressure column configured to adjust a pressure of the fluid within the at least one organ or tissue.

4. The system of claim 1, further comprising control circuitry configured to:
control the pump to regulate the flow of the fluid; and
control the at least one actuator to move the platform.

5. The system of claim 1, wherein the platform is positioned within the interior cavity of the container, wherein the platform is configured to mechanically support the at least one organ or tissue, and wherein the container is configured to mechanically support the platform.

6. The system of claim 1, wherein the platform is positioned underneath the container, and the platform is configured to mechanically support the container, and wherein the container is configured to mechanically support the at least one organ or tissue.

7. The system of claim 1, wherein the container further comprises an access port configured to allow insertion of a medical device into the interior cavity.

8. The system of claim 7, wherein the access port is positioned on the container to facilitate simulation of at least one of femoral access or radial access to the at least one organ or tissue.

9. The system of claim 1, wherein the at least one environmental condition comprises at least one of a temperature, a humidity, or a fluid level, wherein the fluid level is an amount of fluid in the interior cavity.

10. The system of claim 1, wherein the fluid is transparent.

11. The system of claim 1, wherein the fluid comprises a solution comprising sodium, potassium, chloride, calcium, magnesium sulfate, bicarbonate, phosphate, and glucose.

12. The system of claim 1, wherein the fluid comprises blood.

13. The system of claim 1, further comprising at least one physiological sensor configured to measure at least one physiological condition associated with the at least one organ or tissue.

14. The system of claim 13, wherein the at least one physiological condition comprises at least one of a pulse rate, a pulse depth, a pressure of the fluid, a flow rate of the fluid, or a nerve activity.

15. The system of claim 1, further comprising an electrode, positioned within the interior cavity, configured to deliver electrical signals to at least one nerve associated with the at least one organ or tissue.

16. The system of claim 1, further comprising at least one image sensor configured to collect image data of the at least one organ or tissue.

17. A method of operating a perfusion system, comprising:
generating, by a pump, a flow of a fluid through a fluid circuit, wherein the pump comprises a pump outlet, and wherein the fluid circuit is configured to fluidically couple the pump to at least one organ or tissue such that the fluid flows through the at least one organ or tissue in response to the pump generating the flow of the fluid, wherein the perfusion system comprises a container defining an interior cavity configured to receive at least one organ or tissue, and wherein the container is configured to maintain at least one environmental condition associated with the at least one organ or tissue within the interior cavity; and
moving, by at least one actuator operably coupled to a platform operably coupled to the container, the platform to mimic at least one biological movement associated with the at least one organ or tissue.

18. The method of claim 17, further comprising inserting a medical device into the interior cavity via an access port in the container.

19. The method of claim 17, further comprising measuring the at least one environmental condition using at least one environmental sensor.

20. A system comprising:
a pump configured to generate a flow of a fluid;
a container defining an interior cavity and configured to receive at least one organ or tissue and maintain at least one environmental condition associated with the at least one organ or tissue within the interior cavity,
a fluid circuit configured to fluidically couple the pump to the at least one organ or tissue;
at least one image sensor configured to collect image data of the at least one organ or tissue;
a platform operably coupled to the container; and
at least one actuator configured to move the platform to mimic at least one biological movement associated with the at least one organ or tissue.
